SEQUENCE LISTING (18 sequences)

SEQ ID NO: 1 - protein, 761 aa, Homo sapiens
MARRPRHSIYSSDEDDEDFEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTDNAIKNHWNSTMRRKVEQEGYLQESSKASQPAVATSFQKNSHLMGFAQAPPTAQLPATGQPTVNNDYSYYHISEAQNVSSHVPYPVALHVNIVNVPQPAAAAIQRHYNDEDPEKEKRIKELELLLMSTENELKGQQVLPTQNHTCSYPGWHSTTIADHTRPHGDSAPVSCLGEHHSTPSLPADPGSLPEESASPARCMIVHQGTILDNVKNLLEFAETLQFIDSDSSSWCDLSSFEFFEEADFSPSQHHTGKALQLQQREGNGTKPAGEPSPRVNKRMLSESSLDPPKVLPPARHSTIPLVILRKKRGQASPLATGDCSSFIFADVSSSTPKRSPVKSLPFSPSQFLNTSSNHENSDLEMPSLTSTPLIGHKLTVTTPFHRDQTVKTQKENTVFRTPAIKRSILESSPRTPTPFKHALAAQEIKYGPLKMLPQTPSHLVEDLQDVIKQESDESGIVAEFQENGPPLLKKIKQEVESPTDKSGNFFCSHHWEGDSLNTQLFTQTSPVADAPNILTSSVLMAPASEDEDNVLKAFTVPKNRSLASPLQPCSSTWEPASCGKMEEQMTSSSQARKYVNAFSARTLVM

SEQ ID NO: 2 - protein, 341 aa, Homo sapiens
MTMESGAENQQSGDAAVTEAENQQMTVQAQPQIATLAQVSMPAAHATSSAPTVTLVQLPNGQTVQVHGVIQAAQPSVIQSPQVQTVQSSCKDLKRLFSGTQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSD

SEQ ID NO: 3 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 4 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 5 - protein, 12 aa, artificial
  cell penetrating peptide
RRRQRRKKRGYX

SEQ ID NO: 6 - protein, 10 aa, artificial
  cwll penetrating peptide
RRRRRRRRRX

SEQ ID NO: 7 - protein, 16 aa, artificial
  c-Myb-derived peptide sequence
RIKELELLLMSTENEL

SEQ ID NO: 8 - protein, 8 aa, artificial
  c-Myb-derived peptide sequence, D-amino acids
LENETSML

SEQ ID NO: 9 - protein, 16 aa, artificial
  c-Myb-derived peptide sequence, D-amino acids
LENETSMLLLELEKIR

SEQ ID NO: 10 - protein, 14 aa, artificial
  CREB KID domain sequence
SYRKILNDLSSDAP

SEQ ID NO: 11 - protein, 7 aa, artificial
  CREB KID domain, D-amino acids
NLIKRYS

SEQ ID NO: 12 - protein, 14 aa, artificial
  CREB KID domain, D-amino acids
PADSSLDNLIKRYS

SEQ ID NO: 13 - protein, 26 aa, artificial
  therapeutic peptide
RRRRRRRRRGLENETSMLDNLIKRYS

SEQ ID NO: 14 - protein, 27 aa, artificial
  therapeutic peptide
RRRRRRRRRGLENETSMLDNLIKRYSG

SEQ ID NO: 15 - protein, 27 aa, artificial
  therapeutic peptide
RRRRRRRRRGLENETSMXDNLIKRYSG

SEQ ID NO: 16 - protein, 28 aa, artificial
  therapeutic peptide
RRRQRRKKRGYGLENETSMLLLELEKIR

SEQ ID NO: 17 - protein, 25 aa, artificial
  therapeutic peptide
RRRRRRRRRGPADSSLDNLIKRYSG

SEQ ID NO: 18 - protein, 25 aa, artificial
  therapeutic peptide
RRRRRRRRRGPADSSLDNLIKRYSG

(19) United States
(12) Patent Application Publication
(10) Pub. No.: US 2012/0302503 A1
(43) Pub. Date: Nov. 29, 2012

(54) PEPTIDES FOR PREVENTING OR TREATING A DISEASE OR DISORDER ASSOCIATED WITH CBP OR p300 MISREGULATION, AND METHODS FOR USE AND IDENTIFICATION THEREOF

(75) Inventor: Mark R. Hurtt, Cambridge, MA (US)

(73) Assignee: AML Therapeutics, LLC, Cambridge, MA (US)

(21) Appl. No.: 13/465,925

(22) Filed: May 7, 2012

Related U.S. Application Data
(60) Provisional application No. 61/519,471, filed on May 23, 2011.
(60) Provisional application No. 61/592,245, filed on Jan. 30, 2012.

(51) Int. Cl. A61K 38/16; A61P 7/06; A61P 35/00; C07K 14/00; A61P 35/02

(52) U.S. Cl. 514/13.5; 530/324; 514/19.6; 514/19.3; 514/21.3

(57) ABSTRACT

Described herein are therapeutic peptides composed of a cell penetrating peptide, a peptide derived from the sequence of c-Myb, and a peptide derived from the sequence of CREB, useful for the treatment or prevention of a disease or disorder associated with CBP or p300 misregulation.

### RELATED APPLICATIONS

This application claims the benefit of U.S. provisional application No. 61/519,471, filed May 23, 2011; and U.S. provisional application No. 61/592,245, filed Jan. 30, 2012, the entire contents of each of which are herein incorporated by reference.

### BACKGROUND

The proto-oncogene c-Myb and the cAMP-response element binding protein (CREB) interact with CREB binding protein (CBP) and p300 to regulate downstream genes. The misregulation of downstream genes leads to pathological diseases and disorders including, for example, acute leukemia, chronic leukemia, myeloproliferative disorders, lymphoma, solid tumors or β-hemoglobin disorders such as, for example, sickle cell disease and β-thalassemia.

Targeting domains on CBP and/or p300 that block the interaction of c-Myb and/or CREB can reverse the pathologic effects of c-Myb and/or CREB misregulation, which leads to the misregulation of CBP and/or p300. Although such targeting would be useful for treating or preventing diseases or disorders associated with CBP or p300 misregulation, delivery of therapeutic agents is problematic, as cellular uptake of macromolecular agents that compete for CBP and/or p300 binding has not been feasible.

There is, therefore, a need for therapeutic agents that target CBP and/or p300 binding by c-Myb and/or CREB, wherein the therapeutic agents can be delivered to cells in a practicable and effective manner.

### SUMMARY

Described herein a formulations, compositions and methods for using and identifying compositions for treating a disease or disorder associated with CBP and/or p300 misregulation.

In one embodiment, the disclosure is directed to a formulation comprising a peptide comprising R1 and R2, wherein R1 is a cell penetrating peptide, and R2 is a peptide derived from a group consisting of: the sequence of c-Myb, the sequence of CREB, and a sequence comprising both a peptide sequence derived from the sequence of c-Myb and a sequence derived from the sequence of CREB. In a particular embodiment, R1 comprises a sequence that is about 80% identical to or identical to SEQ ID NO:5 or SEQ ID NO:6. In a particular embodiment, R2 comprises a sequence that is about 80% identical to or identical to a sequence selected from the group consisting of SEQ ID NOS:7-9. In a particular embodiment, R2 comprises a sequence that is about 80% identical to or identical to a sequence selected from the group consisting of SEQ ID NOS:10-12. In a particular embodiment, the peptide comprises a sequence that is about 80% identical to or identical to a sequence selected from the group consisting of: SEQ ID NOS:13-18. In a particular embodiment, the peptide is comprised entirely of D-amino acids. In a particular embodiment, the peptide is comprised of a mix of D-amino acids and L-amino acids. In a particular embodiment, the peptide comprises one or more non-naturally occurring amino acids. In a particular embodiment, the peptide comprises one or more synthetic amino acids. In a particular embodiment, the formulation further comprises one or more pharmaceutically acceptable excipients. In a particular embodiment, R1 occurs at the N- or C-terminus of the peptide. In a particular embodiment, the formulation optionally comprises one or more amino acids at the termini of the peptide and/or between R1 and/or R2. In a particular embodiment, the peptide comprises an amino acid with a C-terminal amide modification.

In one embodiment, the disclosure is directed to a method of treating or preventing a disease or disorder associated with CBP or p300 misregulation, comprising administering a therapeutically effective amount or a formulation comprising a peptide comprising R1 and R2, wherein R1 is a cell penetrating peptide, and R2 is a peptide derived from a group consisting of: the sequence of c-Myb, the sequence of CREB, and a sequence comprising both a peptide sequence derived from the sequence of c-Myb and a sequence derived from the sequence of CREB, or a pharmaceutically acceptable salt thereof. In a particular embodiment, the disease or disorder is selected from the group consisting of: acute leukemia, chronic leukemia, myeloproliferative disorders, lymphoma, solid tumors or β-hemoglobin disorders such as, for example, sickle cell disease and β-thalassemia. In a particular embodiment, the dose is selected from the group consisting of: about 0.01 mg/kg/day to about 100 mg/kg/day; about 0.01 to about 0.03 mg/kg/day; about 0.03 to about 0.1 mg/kg/day; about 0.1 to about 0.3 mg/kg/day; about 0.3 to about 1 mg/kg/day; about 1 to about 3 mg/kg/day; about 3 to about 10 mg/kg/day; about 10 to about 30 mg/kg/day; and about 30 to about 100 mg/kg/day. In a particular embodiment, the dose is selected from the group consisting of: about 0.3 to 3000 mg/m2/day; about 0.3 to about 1 mg/m2/day; about 1 to about 3 mg/m2/day; about 3 to about 10 mg/m2/day; about 10 to about 30 mg/m2/day; about 30 to about 100 mg/m2/day; about 100 to about 300 mg/m2/day; about 300 to about 1000 mg/m2/day; and about 1000 to about 3000 mg/m2/day. In a particular embodiment, R1 comprises a sequence that is about 80% identical to or identical to SEQ ID NO:5 or SEQ ID NO:6. In a particular embodiment, R2 comprises a sequence that is about 80% identical to or identical to a sequence selected from the group consisting of: SEQ ID NOS:7-9. In a particular embodiment, R2 comprises a sequence that is about 80% identical to or identical to a sequence selected from the group consisting of: SEQ ID NOS:10-12. In a particular embodiment, the peptide comprises a sequence that is about 80% identical to or identical to a sequence selected from the group consisting of: SEQ ID NOS:13-18. In a particular embodiment, the peptide is comprised entirely of D-amino acids. In a particular embodiment, the peptide is comprised of a mix of D-amino acids and L-amino acids. In a particular embodiment, the peptide comprises one or more non-naturally occurring amino acids. In a particular embodiment, the peptide comprises one or more synthetic amino acids. In a particular embodiment, the formulation further comprises one or more pharmaceutically acceptable excipients. In a particular embodiment, R1 occurs at the N- or C-terminus of the peptide. In a particular embodiment, the peptide optionally comprises one or more amino acids at the termini of the peptide and/or between R1 and/or R2. In a particular embodiment, the peptide comprises an amino acid with a C-terminal amide modification.

In one embodiment, the disclosure is directed to a kit for treating or preventing a disease or disorder associated with CBP or p300 misregulation comprising one or more doses corresponding to a therapeutically effective amount of a formulation a formulation comprising a peptide comprising R1 and R2, wherein R1 is a cell penetrating peptide, and R2 is a peptide derived from a group consisting of: the sequence of c-Myb, the sequence of CREB, and a sequence comprising both a peptide sequence derived from the sequence of c-Myb and a sequence derived from the sequence of CREB, or a pharmaceutically acceptable salt thereof.

In one embodiment, the disclosure is directed to a method for identifying a peptide for treating or preventing a disease or disorder associated with CBP or p300 misregulation comprising: contacting a cell suitable for a cell proliferation assay with a test peptide comprising a cell penetrating peptide and a c-Myb- or CBP-derived peptide, wherein the test peptide is provided in the culture medium of the cell; determining the cell proliferation capacity of the cell; and comparing the cell proliferation of the cell in contact with the test peptide with the cell proliferation of the cell in the absence of a test peptide, wherein a reduction of cell proliferation in the presence of the test peptide is indicative of the peptide's ability to treat or prevent a disease or disorder associated with CBP or p300 misregulation.

### BRIEF DESCRIPTION OF THE DRAWINGS

FIG. 1 is a listing of the amino acid sequence of human transcriptional activator Myb isoform 1 (NCBI Reference Sequence: NP_{—}001123645.1).

FIG. 2 is a listing of the amino acid sequence of Cyclic AMP responsive element binding protein 1 isoform B (CREB) (NCBI Reference Sequence: NP_{—}604391.1).

FIGS. 3A and 3B are a listing of the amino acid sequence of CREB binding protein (CBP) isoform A (NCBI Reference Sequence: NP_{—}004371.2).

FIGS. 4A and 4B are a listing of the amino acid sequence of Histone Acetyltransferase p300 (NCBI Reference Sequence: NP_{—}001420.2).

### DETAILED DESCRIPTION

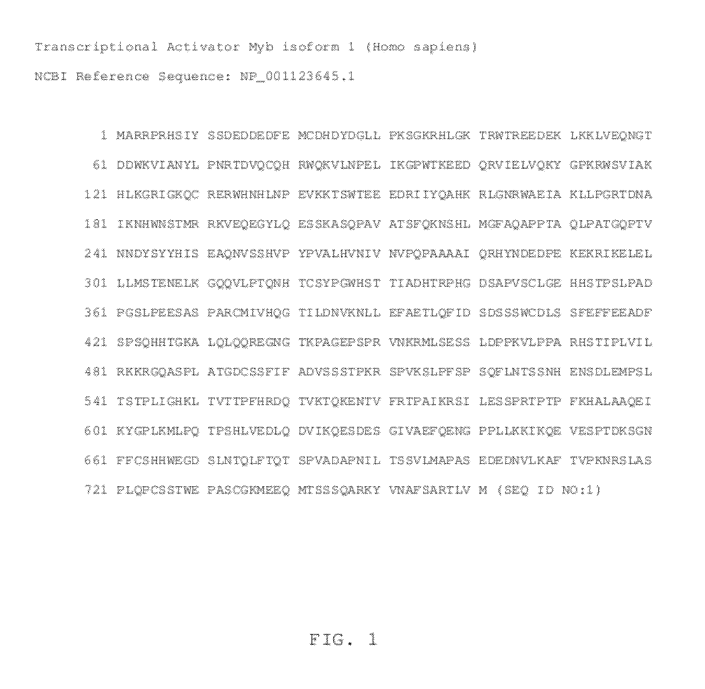

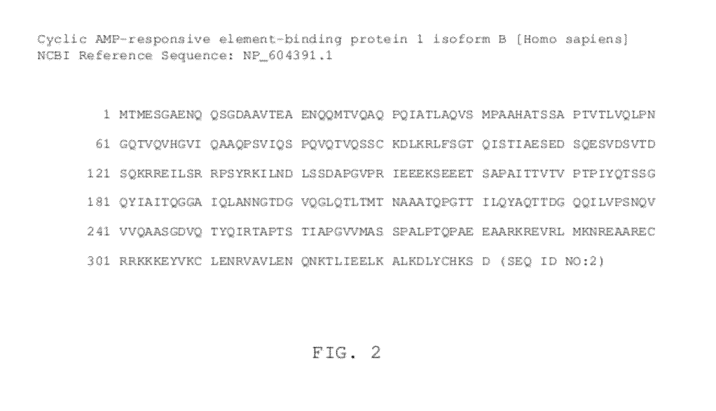

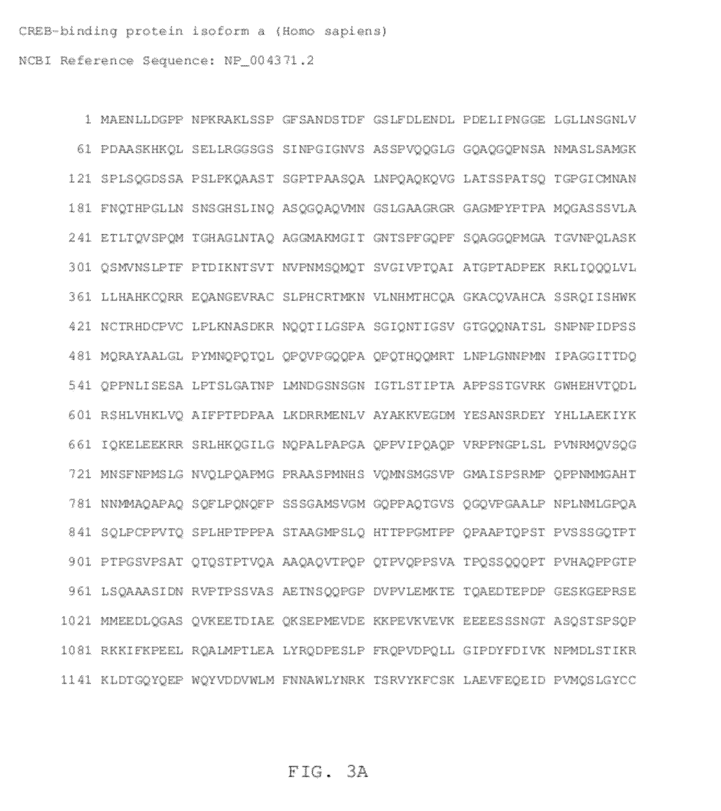

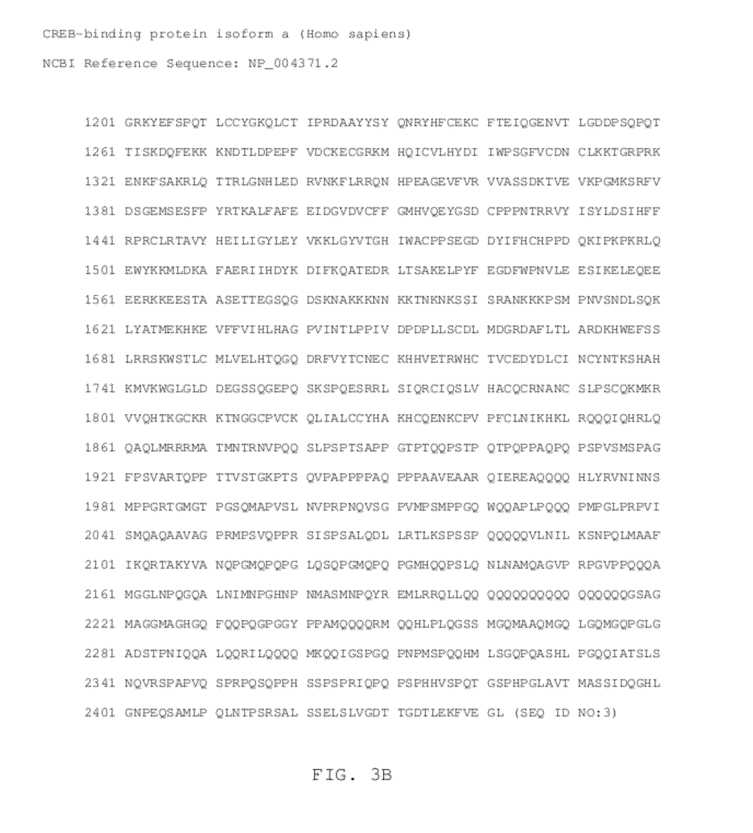

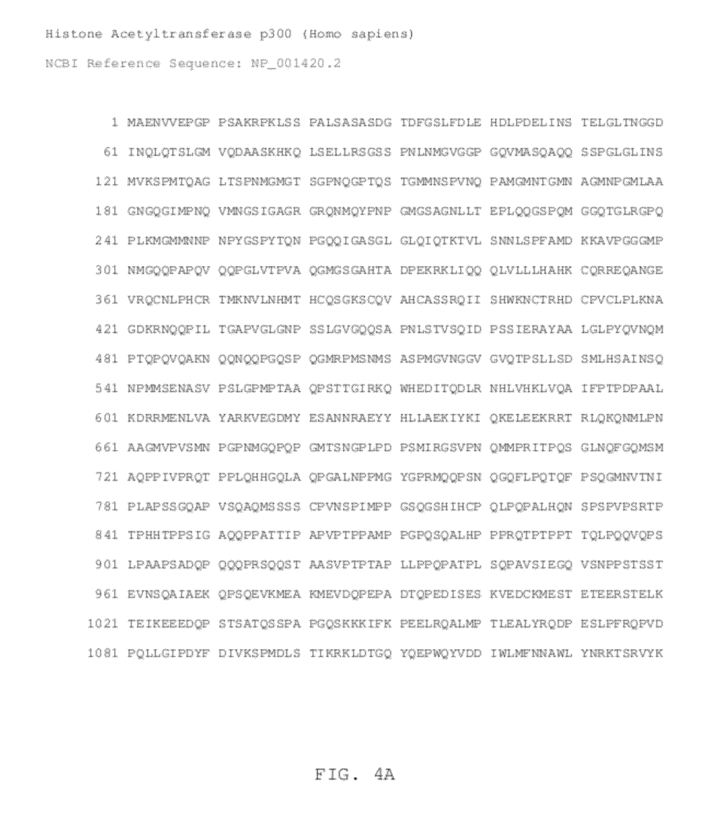

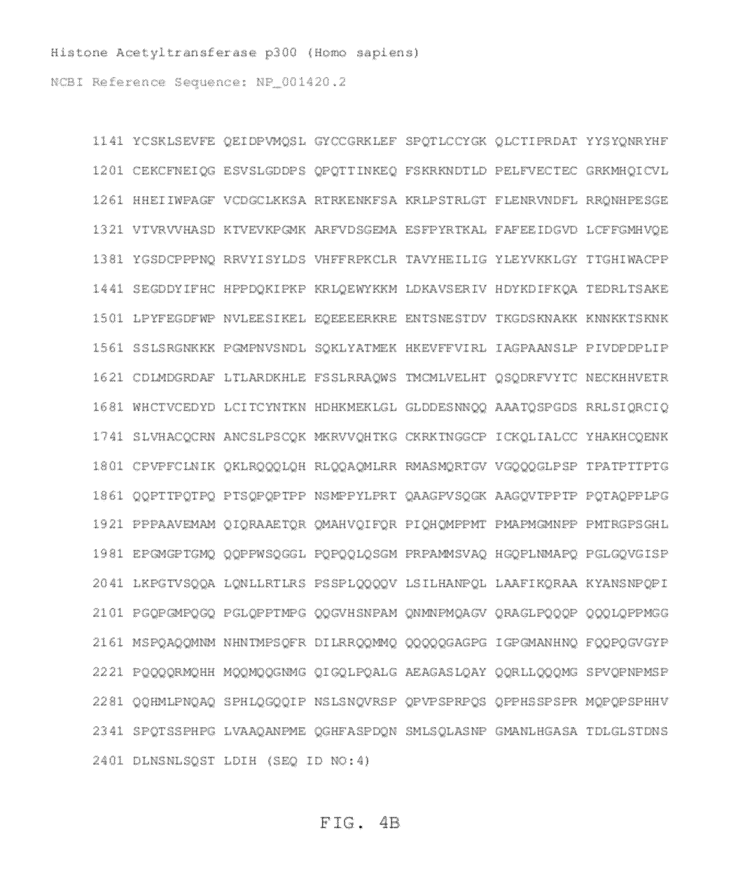

Therapeutic peptides comprising a cell penetrating peptide and one or more peptides derived from c-Myb (FIG. 1) or CREB (FIG. 2), and formulations thereof, are described. The peptides described herein are useful for the treatment and/or prevention of a disease or disorder associated with CREB Binding Protein (CBP) or p300 misregulation (e.g., acute leukemia, chronic leukemia, myeloproliferative disorders, lymphoma, solid tumors or β-hemoglobin disorders such as, for example, sickle cell disease and β-thalassemia), as the peptides interact with CBP (FIG. 3) and/or p300 (FIG. 4) to inhibit the downstream signaling. Methods for identifying such peptides, specific examples of such peptides, methods for using such peptides for the treatment or prevention of a disease or disorder associated with CBP or p300 misregulation and kits utilizing such peptides and methods are disclosed herein.

CBP and p300 have similar structures. Both contain five protein interaction domains: the nuclear receptor interaction domain (RID), the CREB and c-Myb interaction domain (KIX), the cysteine/histidine regions (TAZ1/CH1 and TAZ2/CH3) and the interferon response binding domain (IBiD). The conserved domains are connected by long stretches of unstructured linkers. The peptides described herein bind and inhibit downstream signaling of CBP and/or p300, and are, therefore, useful for the treatment of diseases or disorders associated with CBP or p300 misregulation.

CREB and c-Myb comprise binding domains that are structurally similar and bind to the KIX domains of CBP and p300. The peptides described and identified herein bind to the KIX domains of CBP and/or p300, and inhibit downstream signaling of those effectors. While not wishing to be bound by theory, signaling inhibition can occur through competitive inhibition and/or allosteric inhibition of one or more CBP or p300 active sites.

The compounds provided comprise a peptide described by the general formula, R1-R2, where R1 is a cell penetrating peptide, R2 is a peptide derived from the sequence of c-Myb, CREB or a combination of both c-Myb and CREB. The peptides can optionally contain additional sequences, either on the N- or C-termini of R1-R2, or additional sequences between R1 and R2. Where the peptide comprises additional amino acids at the C-terminus, for example, the terminal amino acid can be modified (e.g., amidation). The position, order, and presence of R1 and R2 can vary, depending on the specific requirements of the compound, as would be determined by one of skill in the art. CREB and c-Myb sequences in R2, for example, can be both present (either in single or multiple copies) or they can be present each in multiple copies. If, for example, R2 comprises multiple c-Myb sequences, multiple CREB sequences, or multiple c-Myb and CREB sequences, the overall peptide can comprise additional spacer sequences between each c-Myb and/or CREB sequence.

### Peptides

The compounds and formulations described herein comprise a peptide comprising various peptide sequences, e.g., peptide sequences “derived” from longer polypeptide sequences. As used herein, a peptide sequence is “derived” from a longer polypeptide sequence if the derived peptide sequence is contained, wholly or partially, within the longer peptide sequence. A derived peptide can be, for example, 3 to 10 amino acids in length, 5 to 20 amino acids in length, 10 to 50 amino acids in length, or longer or any length contained within the described ranges. A derived peptide can be, for example, identical to the polypeptide sequence from which it is derived, about 95% identical to the polypeptide sequence from which it is derived, about 90% identical to the polypeptide sequence from which it is derived, about 85% identical to the polypeptide sequence from which it is derived, about 80% identical to the polypeptide sequence from which it is derived or about 75% identical to the polypeptide sequence from which it is derived. As used herein, the term “about” means plus or minus 10% of the numerical value of the number with which it is being used. About 50%, for example, means in the range of 45%-55%. Alternatively, a derived peptide can comprise amino acids at specific positions within the polypeptide sequence from which it is derived, provided the amino acids at the specific positions are determined to be important for a particular function, e.g., protein-protein interactions, nucleotide or polynucleotide binding, or enzymatic function. In such situations, a derived peptide might have little that is identical to the polypeptide from which it is derived other than the conserved amino acids at positions critical for function.

The terms “peptide” and “polypeptide” are used interchangeably and refer to a polymeric form of amino acids of any length as described above, which can include coding and non-coding amino acids in situations where a peptide or polypeptide is expressed using transcription/translation, chemically or biochemically modified or derivatized amino acids, enantiomeric diverse amino acids within a peptide chain, and peptides comprising modified peptide backbones. The term includes, for example, fusion proteins, including, but not limited to, fusion proteins with a heterologous amino acid sequence, fusions with heterologous and homologous leader sequences, with or without N-terminal methionine residues; immunologically tagged proteins; and the like (e.g., Fields, G. and Noble, R. Int. J. Peptide, Res., 35:161-214, 1990). The peptides described herein can also incorporate, for example, non-hydrolyzable phosphoserine mimetics (e.g., phosphonomethylene alanine or phosphonodifluoromethylene alanine) to substitute for phosphoserine and improve the biological activity of phosphopeptides (Zheng, W. et al., Nat. Struct. Biol., 10:1054-7, 2003; Zheng, W. et al., J. Biol. Chem., 280:10462-7, 2005).

A sequence is said to be “optimized” if it has been altered, for example, for a nucleotide sequence to encode an amino acid sequence using codons that are preferred in the production cell or organism, a peptide sequence to contain amino acid residues (either naturally occurring residues or non-naturally occurring residues) that, for example, increase the stability of the peptide or increase the functional efficacy of the peptide. Retro-inverso isomerization of L-amino acid peptides to reduce hydrolysis and improve their therapeutic activity has been described (e.g., Snyder, E. et al., PLoS Biol., 2:E36, 2004). A retro-inverso peptide is made up of D-amino acids in a reversed sequence and, when extended, assumes a side chain topology similar to that of its parent molecule but with inverted amide peptide bonds (Li, C. et al., J. Biol. Chem., 285:19572-81, 2010). Additionally, substitution of alpha-amino acids with their beta-amino acid homologs can be used to reduce peptide hydrolysis and improve therapeutic activity (Hintersteiner, M. et al., Chembiochem., 10:994-8, 2009; Horne, W. et al., Angew Chem Int Ed Engl., 47:2853-6, 2008; Horne, W. et al., Proc. Natl. Acad. Sci. USA, 106:14751-6, 2009; Sadowsky, J. et al., Chembiochem., 8:903-16, 2007; Seebach, D. et al., Chem. Biodivers., 1:1111-239, 2004).

A “non-naturally occurring amino acid” refers to an amino acid that is not one of the 20 common amino acids. Other terms that may be used synonymously with the term include “non-natural amino acid,” “non-naturally encoded amino acid,” and variously hyphenated and non-hyphenated versions thereof. The term also includes, but is not limited to, modified amino acids (e.g., post-translational modifications, chemical modifications to amino acid side chains or to the peptide backbone, terminally modified amino acids, etc.).

An “amino terminus modification group” refers to any molecule that can be attached to the amino terminus of a polypeptide. Similarly, a “carboxy terminus modification group” refers to any molecule that can be attached to the carboxy terminus of a polypeptide. Terminus modification groups include, but are not limited to, chemical modification groups (e.g., amide modification), various water soluble polymers, peptides or proteins such as serum albumin, or other moieties that, for example, increase serum half-life of peptides and/or the efficacy of the peptides.

Optimized sequences are engineered, for example, to encode an amino acid sequence identical or nearly identical to a starting amino acid sequence, which is also known as the “parental” sequence. One of skill in the art will recognize that conservative substitutions can be made to peptide sequences that result in functional or optimized peptides. The disclosure provides for the use of sequences that are at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to desired parental sequences. The terms “homology” or “identity” or “similarity” refer to sequence relationships between sequences and can be determined by comparing a position in each sequence when aligned for purposes of comparison. The term “homology” refers to the relatedness of two nucleic acid or protein sequences. The term “identity” refers to the degree to which amino acids are the same between two sequences. The term “similarity” refers to the degree to which amino acids are the same.

One of ordinary skill in the art will recognize that individual substitutions, deletions or additions to a nucleic acid, peptide, polypeptide, or protein sequence that alter, add or delete a single amino acid or a small percentage of amino acids in the encoded sequence is a “conservatively modified variant.” Such variants can be useful, for example, to alter the physical properties of the peptide, e.g., to increase stability or efficacy of the peptide. Conservative substitution tables providing functionally similar amino acids are known to those of ordinary skill in the art. Such conservatively modified variants are in addition to and do not exclude polymorphic variants, interspecies homologs and alternate alleles. The following eight groups provide non-limiting examples of amino acids that can be conservatively substituted for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Peptides described herein can optionally comprise conjugates of substances having a wide variety of functional groups, substituents or moieties, with other substances including but not limited to a label; a dye; a polymer; a water-soluble polymer; a derivative of polyethylene glycol; a photocrosslinker; a radionuclide; a cytotoxic compound; a drug; an affinity label; a photoaffinity label; a reactive compound; a resin; an additional protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or non-covalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance.

The peptides described herein can be synthesized by methods known in the art, or they can be produced by transcription/translation systems known in the art (both in vivo and in vitro systems). For such expression systems, the present disclosure also provides vectors (e.g., plasmid, phage, expression), cell lines (e.g., mammalian, insect, yeast, bacterial), and kits comprising any of the sequences of the disclosure described herein, as would be determined by one of skill in the art according to the expression system to be used.

The compounds of this disclosure can exist in radiolabeled form, i.e., the compounds can contain one or more atoms containing an atomic mass or mass number different from the atomic mass or mass number most commonly found in nature. Radioisotopes of, for example, hydrogen, carbon, phosphorous, fluorine, and chlorine include ²H, ³H, ¹³C, ¹⁴C, ¹⁵N, ³⁵S, ¹⁸F and ³⁶Cl, respectively. Compounds that contain those radioisotopes and/or other radioisotopes of other atoms are within the scope of this disclosure. Radiolabeled compounds of the present disclosure and prodrugs thereof can generally be prepared by methods well known to those skilled in the art.

The compounds described herein can contain asymmetric centers and can thus give rise to enantiomers, diastereomers and other stereoisomeric forms, either as a whole or at specific diastereomeric centers. The present disclosure is meant to include all such possible isomers, as well as, their racemic and optically pure forms, e.g., D- and L-amino acids within a peptide sequence. The single letter amino acid code is used throughout this specification, where the letter “X” designates the suitability of any amino acid, and the letters “pS” designate the suitability of phosphoserine, or of a phosphoserine mimetic amino acid such as phosphonomethylene alanine or phosphonodifluoromethylene alanine. The phrase “all D-isomer amino acids” applies only to the chiral amino acids of a peptide; glycine is recognized as being achiral. If not indicated, the single-letter code for an amino acid represents the L-form of the amino acid. D-amino acids are denoted by a lower case “d” immediately before the single letter amino acid designation. Modified amino acids are so indicated with an appropriate modifier to the single-letter code (e.g., “pS” refers to phospho-L-serine, and “pdS” refers to phospho-D-serine). Isomers of phosphoserine (pS and dpS) can be replaced, for example, by isomers of phosphonomethylene alanine (pmA and dpmA) and/or isomers of phosphonodifluoromethylene alanine (pfA and dpfA). Modifications to the termini of a peptide sequence are denoted as appropriate, e.g., a C-terminal amide modification is denoted by the word “amide” at the C-terminus. Variable or substitutable positions are denoted with the letter “X” and can refer to either a single amino acid, a peptide sequence, or a chemical moiety, as indicated.

Cell Penetrating Peptides

Cell-penetrating peptides facilitate cellular uptake of, for example, other peptides. The delivery function of the cell penetrating peptides commonly occurs through endocytosis, with the cargo delivered to the endosomes of cells. Cell penetrating peptides typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. The use of cell penetrating peptides for intracellular delivery of peptides has been reviewed, for example, by Heitz, F. et al. (Br. J. Pharmacol., 157:195-206, 2009). Cell penetrating peptides (also known as protein transduction domains, or PTDs) based on the TAT protein of HIV (U.S. Pat. Nos. 5,652,122; 5,670,617; 5,674,980 and 5,804,604), and cell penetrating peptides based on polyarginine are claimed in U.S. Pat. Nos. 6,306,993 and 6,495,663) have been described.

Described herein are compounds and formulations comprising one or more peptides comprising a cell penetrating peptide sequence. The cell penetrating peptide sequence can occur anywhere within the overall peptide, however the peptides described herein comprise a terminal cell penetrating peptide, for example, an N-terminal or a C-terminal cell penetrating peptide. Additionally, the peptide can comprise more than one cell penetrating peptide sequence. One of skill in the art would determine the optimal position(s) of the cell penetrating peptide(s) to confer delivery of the peptide into a cell.

The cell penetrating peptide, R1, can be, for example either the peptide composed of all D-isomer amino acids dR dR dR dQ dR dR dK dK dR G dY X (SEQ ID NO:5), wherein “X” can be, for example, a modified amino acid, e.g., an amidated glycine; or the peptide composed of all D-isomer amino acids dR dR dR dR dR dR dR dR dR X (SEQ ID NO: 6), wherein “X” can be the C-terminus, a single amino acid, or a peptide; or a combination of D- or L-amino acids within these and/or other cell penetrating peptide sequences. If retro-inverso sequences are used, D-amino acids can be used exclusively, or they can be used in combination with L-amino acids, where the D-amino acids are in inverse order and the L-amino acids are in there normal sequence.

C-Myb-Derived Peptides

C-Myb is a proto-oncogene that acts as a transcription factor that regulates hematopoietic cell proliferation and differentiation (Grieg, K. et al., Sem. Immunol., 20:247-56, 2008; Lieu, Y. & Reddy, E., Proc. Natl. Acad. Sci. USA, 106:21689-94, 2009). Activating mutations of c-Myb cause leukemia, and inactivating mutations of c-Myb impair hematopoesis and leukemogenesis (Pattabiraman, D. et al., Mol. Cancer. Res., 7:1477-86, 2009). C-Myb is a molecular target for discovery and development of compounds to treat leukemia, lymphoma or solid tumors (Ramsay, R. & Gonda, T., Nat. Rev. Cancer, 8:523-34, 2008). C-Myb activates transcription of its target genes via interactions with the transcriptional co-activators CBP and p300 (Goodman, R. & Smolik, S., Genes Dev., 14:1553-1577, 2000). C-Myb regulates fetal hemoglobin (HbF), and increased HbF can ameliorate β-hemoglobin disorders such as, for example, sickle cell disease and β-thalassemia (Sankaran, V., Hematology, 459-65, 2011).

CBP and p300 share a common interaction domain, KIX, which interacts with the transactivation domain (TAD) of c-Myb and the kinase-inducible domain (KID) of CREB (cyclic AMP response element binding protein; Zor, T. et al., J. Mol. Biol., 337:521-34, 2004).

The proto-oncogene c-Myb is a member of the Myb gene family, which also includes A-Myb and B-Myb. C-Myb was identified as the cellular counterpart of the transforming v-Myb gene carried by the AMV and E26 retroviruses, both of which induce leukemias in chickens (Baluda, M. & Reddy, E., Oncogene, 9:2761-2774, 1994). Homozygous c-Myb-null mice die at about embryonic day 15 during development as a result of a failure to transition from fetal to adult erythropoiesis (Mucenski, M. et al., Cell, 65:677-689, 1991). Most of what is known about the gene comes from cell lines, which implicate a role for c-Myb in cell proliferation, survival, and/or differentiation.

The c-Myb protein contains three functional domains: the DNA-binding domain (DBD), the transactivation domain (TAD), and the negative regulatory domain (NRD)(Sakura, H. et al., Proc. Natl. Acad. Sci. USA, 86:5758-62, 1989). The DBD and TAD are essential for c-Myb function and for transformation; hence, it is likely that the protein carries out its normal and transforming functions by regulating the expression of other cellular genes. The NRD contains multiple subdomains, including a putative leucine zipper motif/heptad leucine repeat.

For the peptides described herein, a c-Myb-derived peptide sequence, R2, can comprise, for example, amino acids RIKELELLLMSTENEL (SEQ ID NO:7), representing amino acids 294 to 309 of the c-Myb sequence, according to the numbering system used by Zor, T. et al. (J. Mol. Biol., 337:521-34, 2004), or any peptide based on the c-Myb sequence. The binding sequence of c-Myb can be used in inverse order, for example, using D-amino acids or a combination of D-amino acids and L-amino acids. Specifically, the amino acid sequence dL dE dN dE dT dS dM dL (SEQ ID NO:8), representing amino acids 309 to 302 of the c-Myb sequence, or dL dE dN dE dT dS dM dL dL dL dE dL dE dK dI dR (SEQ ID NO:9), representing amino acids 309 to 294 of the c-Myb sequence, can be used as D-amino acids in reverse sequence order. R2 can, for example, comprise D-amino acids, L-amino acids, or a combination of D- and L-amino acids. R2, additionally, can comprise naturally occurring amino acids, non-naturally occurring amino acids, or a combination of the two.

CREB-Derived Peptides

CREB (cAMP response element-binding) is a cellular transcription factor. It binds to certain DNA sequences called cAMP response elements (CRE), thereby increasing or decreasing the transcription of downstream genes, e.g., c-fos, the neurotrophin BDNF (Brain-derived neurotrophic factor), tyrosine hydroxylase, and neuropeptides (such as, for example, somatostatin, enkephalin, VGF, and corticotropin-releasing hormone). CREB functions by binding to CREB binding protein (CBP) to regulate transcription. The binding domains (KID) of CREB that interact with CBP show some similarity to the c-Myb domain that interacts with CBP. Domains from CREB or c-Myb that interact with CBP can be used as described herein to bind CBP and/or p300. Such domains can be optimized by one of skill in the art, for example, to strengthen interactions with CBP and/or p300.

The KID domain of CREB interacts with the KIX domain of CBP. The CREB KID domain contains the sequence, SYRKILNDLSSDAP (SEQ ID NO:10), which corresponds to amino acid positions 133 to 146 of CREB according to the numbering of Zor et al. Peptides derived from CREB can contain this sequence or the inverse sequence is D-amino acids are used. For the peptides described herein, R2 can be a peptide comprising, for example, dD dN dL dI dK dR dY pS (SEQ ID NO:11), representing amino acids 140 to 133 of the CREB sequence with a modified phosphoserine; dP dA dD dS dS dL dD dN dL dI dK dR dY pS (SEQ ID NO:12), corresponding to amino acids 146 to 133 of the CREB sequence with a modified phosphoserine; or any other peptide based on the CREB sequence. When used in reverse order, e.g., SEQ ID NOS:11 and 12, the peptides can comprise all D-amino acids or a combination of D- and L-amino acids. In particular, the terminal phosphoserine can be either a D-amino acid or an L-amino acid irrespective of the stereochemistry of the rest of the amino acids in the peptide sequence. R2 CREB sequences can comprise naturally occurring amino acids, non-naturally occurring amino acids, or a combination of the two.

Additional Peptide Sequences

The formulations described herein comprise a peptide with one or more peptide sequences, e.g., R1 and R2. These peptide sequences can occur in any order within the peptide, and the peptide can also comprise additional sequences, for example, between R1 and R2, between sequences in R2, or at the termini of the peptide. The additional sequences can be, for example, “spacer” sequences that align R1 and R2 at a particular physical distance to, for example, improve CBP and/or p300 binding. The additional sequence(s) can also provide stability of the peptide, e.g., protection from cellular degradation. The additional sequences can also provide additional functionality, e.g., transcriptional regulation, cellular localization signals, enzymatic activity, and or molecular binding sites.

Spacer and terminal additional sequences refer to one or more optional peptide sequences and may be any amino acid with or without a C-terminal amide modification. They can, for example, comprise D-amino acids, L-amino acids, or a combination of D- and L-amino acids. Spacer and additional sequences, additionally, can comprise naturally occurring amino acids, non-naturally occurring amino acids, or a combination of the two.

### Methods of Treating

The formulations, compounds and peptides described herein are useful for the treatment of diseases or disorders induced, for example, by c-Myb or CREB signaling through CBP or p300. The diseases or disorders associated with CBP or p300 misregulation include, for example, acute leukemia, chronic leukemia, myeloproliferative disorders, lymphoma, solid tumors or β-hemoglobin disorders such as, for example, sickle cell disease and β-thalassemia.

“Treatment” refers to the administration of medicine or the performance of medical procedures with respect to a patient or subject, for either prophylaxis (prevention) or to cure or reduce the symptoms of the infirmity or malady in the instance where the patient is afflicted. The formulations and compounds described herein or identified through methods described herein can be used as part of a treatment regimen in therapeutically effective amounts. A “therapeutically effective amount” is an amount sufficient to decrease, prevent or ameliorate the symptoms associated with a medical condition. The present disclosure, for example, is directed to treatment using a therapeutically effective amount of a compound sufficient to treat a disease or disorder associated with CBP or p300 misregulation. The terms “patient” and “subject” mean all mammals including humans.

Administration of the formulations or compounds described herein for treating a patient or subject can be by any of the routes normally used for introducing a macromolecule, e.g., a peptide or a compound or formulation comprising a peptide, into ultimate contact with blood or tissue cells. Suitable methods of administering such polypeptides to a patient are available, and, although more than one route can be used to administer a particular composition, a particular route can often provide a more immediate and more effective action or reaction than another route, as would be determined by one of skill in the art. Delivery methods can include, for example, parenteral injections or infusions, transdermal iontophoresis, oral delivery (including the use of microspheres), nasal delivery, ocular delivery, rectal delivery, buccal delivery, delivery using microcapsules, delivery using liposomes, and/or delivery using transdermal patches (including the use of microneedles). Pharmaceutically acceptable carriers are determined in part by the particular composition being administered, as well as by the particular method used to administer the composition. Accordingly, there is a wide variety of suitable formulations of pharmaceutical compositions, as would be known to one of skill in the art.

Formulations suitable for parenteral administration, such as, for example, by intraarticular (in the joints), intravenous, intramuscular, intradermal, intraperitoneal, and subcutaneous routes, can include aqueous and non-aqueous, isotonic sterile injection solutions, which can contain, for example, antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Buffer solutions can be determined, for example, by one of skill in the art to increase stability of the peptide agent in the patient, or to control the release of the peptide agent during treatment. Formulations are also determined for continuous infusion delivery methods.

Injectable depot formulations can be used to deliver peptide therapeutic agents. Such depot formulations can comprise, for example, sustained-release carriers, typically polymers. Sustained-release carriers for delivery include, for example, non-degradable crosslinked polyacrylamide and polyvinylpyrrolidone, poly[lactide-co-glycolide], hydroxyethylmethacrylate and ethylene-vinyl acetate, poly[lactic acids], poly[glycolic acids], polydioxanes, polylactones, polyester hydrogels and polyoxalates. Additional examples of carriers are described, for example, by Holland, J. and Tigh, B. (J. Controlled Release, 4:155-180, 1986), the entire contents of which are hereby incorporated by reference.

The treatment(s) described herein are understood to utilize formulations including compounds identified herein or identified through methods described herein and, for example, salts, solvates and co-crystals of the compound(s). The compounds of the present disclosure can exist in unsolvated as well as solvated forms with pharmaceutically acceptable solvents such as, for example, water, ethanol and the like. In general, the solvated forms are considered equivalent to the unsolvated forms for the purposes of the present disclosure.

The term “pharmaceutically acceptable salts, esters, amides and prodrugs” as used herein refers to those carboxylate salts, amino acid addition salts, esters, amides, prodrugs and inclusion complexes of the compounds of the present disclosure that are, within the scope of sound medical judgment, suitable for use in contact with the tissues of patients without undue toxicity, irritation, allergic response and the like, commensurate with a reasonable benefit/risk ratio, and effective for their intended use, as well as the zwitterionic forms, where possible, of the compounds of the disclosure.

The term “prodrug” refers to compounds that are rapidly transformed in vivo to yield the parent compounds of the above formula, for example, by hydrolysis in blood (T. Higuchi and V. Stella, “Pro-drugs as Novel Delivery Systems,” Vol. 14 of the A.C.S. Symposium Series; Bioreversible Carriers in Drug Design, ed. Edward B. Roche, American Pharmaceutical Association and Pergamon Press, 1987; both of which are incorporated herein by reference in their entirety). Activation in vivo may come about by chemical action or through the intermediacy of enzymes.

The term “solvate” refers to a compound in the solid state, wherein molecules of a suitable solvent are incorporated. A suitable solvent for therapeutic administration is physiologically tolerable at the dosage administered. Examples of suitable solvents for therapeutic administration include, for example, ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Co-crystals are combinations of two or more distinct molecules arranged to create a unique crystal form whose physical properties are different from those of its pure constituents (Remenar, J. et al., J. Am. Chem. Soc., 125:8456-8457, 2003). Inclusion complexes are described in Remington: The Science and Practice of Pharmacy 19.sup.th Ed. (1995) volume 1, page 176-177. Commonly employed inclusion complexes are those with cyclodextrins, and all cyclodextrin complexes, natural and synthetic, with or without added additives and polymer(s), as described in U.S. Pat. Nos. 5,324,718 and 5,472,954. The disclosures of Remenar, Remington and the '718 and '954 patents are incorporated herein by reference in their entireties.

The compounds can be presented as salts. The term “pharmaceutically acceptable salt” refers to salts whose counter ion derives from pharmaceutically acceptable non-toxic acids and bases. Suitable pharmaceutically acceptable base addition salts for the compounds of the present disclosure include, but are not limited to, metallic salts made from aluminum, calcium, lithium, magnesium, potassium, sodium and zinc or organic salts made from lysine, N,N-dialkyl amino acid derivatives (e.g., N,N-dimethylglycine, piperidine-1-acetic acid and morpholine-4-acetic acid), N,N′-dibenzylethylenediamine, chloroprocaine, choline, diethanolamine, ethylenediamine, meglumine (N-methylglucamine) and procaine. Where the compounds contain a basic residue, suitable pharmaceutically acceptable base addition salts for the compounds include, for example, inorganic acids and organic acids. Examples include acetate, benzenesulfonate (besylate), benzoate, bicarbonate, bisulfate, carbonate, camphorsulfonate, citrate, ethanesulfonate, fumarate, gluconate, glutamate, bromide, chloride, isethionate, lactate, maleate, malate, mandelate, methanesulfonate, mucate, nitrate, pamoate, pantothenate, phosphate, succinate, sulfate, tartrate, p-toluenesulfonate, and the like (Barge, S. et al., 1977. J. Pharm. Sci., 66:1-19, the entire contents of which are incorporated herein by reference).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. Solutions and suspensions can be prepared from sterile powders, granules, and tablets of the kind previously described.

Freeze-drying is a commonly employed technique for presenting peptides. The process serves to remove water from the peptide formulation of interest. Freeze-drying, or lyophilization, is a process by which the material to be dried is first frozen and then the ice or frozen solvent is removed by sublimation in a vacuum environment. An excipient can be included in pre-lyophilized formulations to enhance stability during the freeze-drying process and/or to improve stability of the lyophilized product upon storage.

The spray drying of pharmaceuticals is also known to those of ordinary skill in the art. In addition to small molecule pharmaceuticals, a variety of biological materials have been spray dried and these include: enzymes, sera, plasma, micro-organisms and yeasts. Spray drying is a useful technique because it can convert a liquid pharmaceutical preparation into a fine, dustless or agglomerated powder in a one-step process. The basic technique comprises the following four steps: a) atomization of the feed solution into a spray; b) spray-air contact; c) drying of the spray; and d) separation of the dried product from the drying air. U.S. Pat. Nos. 6,235,710 and 6,001,800, which are incorporated by reference herein in their entireties, describe, for example, the preparation of recombinant erythropoietin by spray drying.

The pharmaceutical compositions of the invention can comprise a pharmaceutically acceptable carrier, excipient or stabilizer. Pharmaceutically acceptable carriers are determined in part by the particular composition being administered, as well as by the particular method used to administer the composition. Accordingly, there is a wide variety of suitable formulations of pharmaceutical compositions. Suitable carriers include, but are not limited to, buffers containing succinate, phosphate, borate, HEPES, citrate, histidine, imidazole, acetate, bicarbonate, and other organic acids; antioxidants including but not limited to, ascorbic acid; low molecular weight polypeptides including but not limited to those less than about 10 residues; proteins, including but not limited to, serum albumin, gelatin, or immunoglobulins; hydrophilic polymers including but not limited to, polyvinylpyrrolidone; amino acids including but not limited to, glycine, glutamine, asparagine, arginine, histidine or histidine derivatives, methionine, glutamate, or lysine; monosaccharides, disaccharides, and other carbohydrates, including but not limited to, trehalose, sucrose, glucose, mannose, or dextrins; chelating agents including but not limited to, EDTA and edentate disodium; divalent metal ions including but not limited to, zinc, cobalt, or copper; sugar alcohols including but not limited to, mannitol or sorbitol; salt-forming counter ions including but not limited to, sodium and sodium chloride; and/or nonionic surfactants including but not limited to Tween (including but not limited to, Tween 80 (polysorbate 80) and Tween 20 (polysorbate 20), Pluronics and other pluronic acids, including but not limited to, and other pluronic acids, including but not limited to, pluronic acid F68 (poloxamer 188), or PEG. Suitable surfactants include for example but are not limited to polyethers based upon poly(ethylene oxide)-poly(propylene oxide)-poly(ethylene oxide), e.g., (PEO-PPO-PEO), or poly(propylene oxide)-poly(ethylene oxide)-poly(propylene oxide), e.g., (PPO-PEO-PPO), or a combination thereof. PEO-PPO-PEO and PPO-PEO-PPO are further described in U.S. Pat. No. 4,820,352 incorporated herein in its entirety by reference.

Other ethylene/polypropylene block polymers may be suitable surfactants. A surfactant or a combination of surfactants may be used to stabilize active agents against one or more stresses including, but not limited to, stress that results from agitation. Some of the above may be referred to as “bulking agents.” Some may also be referred to as “tonicity modifiers.” Antimicrobial preservatives can also be applied for product stability and antimicrobial effectiveness; suitable preservatives include, but are not limited to, benzyl alcohol, bezalkonium chloride, metacresol, methyl/propyl parabene, cresol, and phenol, or a combination thereof.

Diluents that are suitable for use in the pharmaceutical composition of the present disclosure include, for example, pharmaceutically acceptable inert fillers such as microcrystalline cellulose, lactose, sucrose, fructose, glucose dextrose, or other sugars, dibasic calcium phosphate, calcium sulfate, cellulose, ethylcellulose, cellulose derivatives, kaolin, mannitol, lactitol, maltitol, xylitol, sorbitol, or other sugar alcohols, dry starch, saccharides, dextrin, maltodextrin or other polysaccharides, inositol or mixtures thereof. The diluent can be, for example, a water-soluble diluent. Examples of diluents include, for example: microcrystalline cellulose such as Avicel PH112, Avicel PH101 and Avicel PH102 available from FMC Corporation; lactose such as lactose monohydrate, lactose anhydrous, and Pharmatose DCL 21; dibasic calcium phosphate such as Emcompress; mannitol; starch; sorbitol; sucrose; and glucose. Diluents are carefully selected to match the specific composition with attention paid to the compression properties. The diluent can be used in an amount of about 2% to about 80% by weight, about 20% to about 50% by weight, or about 25% by weight of the treatment formulation.

Other agents that can be used in the treatment formulation include, for example, a surfactant, dissolution agent and/or other solubilizing material. Surfactants that are suitable for use in the pharmaceutical composition of the present disclosure include, for example, sodium lauryl sulphate, polyethylene stearates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxamers, polyvinyl alcohol and sorbitan fatty acid esters. Dissolution agents increase the dissolution rate of the active agent and function by increasing the solubility of the active agent. Suitable dissolution agents include, for example, organic acids such as citric acid, fumaric acid, tartaric acid, succinic acid, ascorbic acid, acetic acid, malic acid, glutaric acid and adipic acid, which may be used alone or in combination. These agents can also be combined with salts of the acids, e.g., sodium citrate with citric acid, to produce a buffer system. Other agents that can be used to alter the pH of the microenvironment on dissolution include salts of inorganic acids and magnesium hydroxide.

Disintegrants that are suitable for use in the pharmaceutical composition and formulations include, for example, starches, sodium starch glycolate, crospovidone, croscarmellose, microcrystalline cellulose, low substituted hydroxypropyl cellulose, pectins, potassium methacrylate-divinylbenzene copolymer, poly(vinyl alcohol), thylamide, sodium bicarbonate, sodium carbonate, starch derivatives, dextrin, beta cyclodextrin, dextrin derivatives, magnesium oxide, clays, bentonite and mixtures thereof.

The active ingredient, e.g., therapeutic peptides, can be mixed with excipients, which are pharmaceutically acceptable and compatible with the active ingredient and in amounts suitable for use in the therapeutic methods described herein. Various excipients can be homogeneously mixed with the active agent of the present disclosure as would be known to those skilled in the art. The active agent, for example, can be mixed or combined with excipients such as but not limited to microcrystalline cellulose, colloidal silicon dioxide, lactose, starch, sorbitol, cyclodextrin and combinations of these.

Formulations described herein can also optionally include other therapeutic ingredients, anti-caking agents, preservatives, sweetening agents, colorants, flavors, desiccants, plasticizers, dyes, and the like.

The pharmaceutical formulations can be provided, for example, in a unit dosage form, and can be suitably packaged, for example, in a box, blister, vial, bottle, sachet, ampoule or in any other suitable single-dose or multi-dose holder or container (which may be properly labeled); optionally with one or more leaflets containing product information and/or instructions for use.

Formulations suitable for oral administration can be presented as discrete units such as capsules, cachets or tablets each containing a predetermined amount of the active ingredient; as a powder (including micronized and nanoparticulate powders) or granules; as a solution or a suspension in an aqueous liquid or a non-aqueous liquid; or as an oil-in-water liquid emulsion or a water-in-oil liquid emulsion.

The treatments (therapies) described herein can also be part of “combination therapies.” Combination therapy can be achieved by administering two or more agents, each of which is formulated and administered separately, or by administering two or more agents in a single formulation. The second active ingredient can be, for example, a second compound identified herein or through screens described herein, or active ingredients useful for treating, for example, a disease or disorder associated with CBP or p300 misregulation, or symptoms associated with treatment by the first active agent (“side effects”). Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days or weeks. Thus, the two or more agents can be administered within minutes of each other or within any number of hours of each other or within any number or days or weeks of each other.

The pharmaceutical compositions of the compounds provided may be formulated to suit a selected route of administration, and may contain ingredients specific to the route of administration. These pharmaceutical compositions may be administered by any appropriate route, but are preferably administered by intravenous, subcutaneous, intramuscular, intraperitoneal, or intrathecal injection or infusion.

Generally, the therapeutically effective dose of the compounds provided, for a human subject, expressed in units related to total body mass are from 0.01 mg/kg/day to 100 mg/kg/day (inclusive). The therapeutically effective dose may be in the range of about 0.01 mg/kg/day to about 100 mg/kg/day; about 0.01 to about 0.03 mg/kg/day; about 0.03 to about 0.1 mg/kg/day; about 0.1 to about 0.3 mg/kg/day; about 0.3 to about 1 mg/kg/day; about 1 to about 3 mg/kg/day; about 3 to about 10 mg/kg/day; about 10 to about 30 mg/kg/day; and about 30 to about 100 mg/kg/day. Alternatively, the therapeutically effective dose of the compounds provided, for a human subject, expressed in units related to total body surface area, are from about 0.3 to about 3000 mg/m²/day (inclusive). The therapeutically effective dose may be in the range of about 0.3 to 3000 mg/m²/day; about 0.3 to about 1 mg/m²/day; about 1 to about 3 mg/m²/day; about 3 to about 10 mg/m²/day; about 10 to about 30 mg/m²/day; about 30 to about 100 mg/m²/day; about 100 to about 300 mg/m²/day; about 300 to about 1000 mg/m²/day; and about 1000 to about 3000 mg/m²/day.

Doses may be given once daily, or in several divided doses daily, or by continuous administration. The duration of treatment may be for less than one minute, one or several minutes, one or several hours, one or several days, one or several weeks, one or several months, or for an extended period of one or several years.

The compounds provided may be concomitantly administered with other therapeutic agents for the treatment of leukemia, lymphoma, or solid tumors, and may be incorporated into chemotherapy regimens involving repetitive cycles of dosing.

### Methods for Identifying Additional Peptides

One of skill in the art would realize that the peptides described herein can be altered to vary their physical properties (e.g., to optimize functionality). Described herein are sufficient examples of polypeptides derived from CBP-binding or p300-binding proteins, e.g., c-Myb and CREB, joined to cell penetrating peptides that are delivered to cells intact. This finding, together with knowledge of the art, demonstrates that peptides derived from c-Myb and/or CREB joined to one or more cell-penetrating peptide sequences, are useful for the treating or preventing of diseases or disorders associated with CBP or p300 misregulation.

One of skill in the art would, for example, be able to modify the specific sequences described herein, and determine the effect of such modification on the functionality of the peptide. One could, for example, assay protein-protein interactions between the modified peptides and CBP and/or p300. A modified peptide would be identified to be suitable for the methods described herein if, for example, it demonstrates enhanced CBP-binding and/or p300 binding.

One of skill in the art could also identify additional peptides using a screen described, for example, in the Example. A cell proliferation assay, wherein, for example, a candidate peptide to be evaluated is introduced into the extracellular medium of a desired cell line, would measure both the uptake of the peptide by the desired cell line as well as the ability of the peptide to inhibit cell proliferation.

The unexpected finding described herein that a peptide can be engineered to be readily delivered across a cell membrane to a cell, wherein the peptide remains functionally intact after translocation, would allow one of skill in the art to use the assays described herein to identify other peptides suitable for use in the treatment methods described herein.

### Kits

The peptides and formulations comprising the peptides described herein can be included, for example, in a kit that comprises a suitable formulation of the active peptide agent and, optionally, devices for administering the therapeutic formulations, buffers and delivery media for administering the therapeutic agents, and instructions for using the kits, including, for example, dosing instructions.

### Example

### Cell Proliferation Assays

To determine the in vitro IC₅₀ of two peptides, and a positive control, cisplatin, against the THP-1 cell line, cell growth is determined using Promega's Cell Titer-Glo® assay.

Method

IC₅₀ Value Determination of Single Agent

THP-1 tumor cells are placed in a 96-well microculture plate (Costar white, flat bottom #3917) at 20,000 cells per well, in a total volume of 90 μL/well. After 24 hours of incubation in a humidified incubator at 37 C with 5% CO₂ and 95% air, 10 μL of 10×, serially diluted test agents in growth medium are added to each well. After 96 total hours of culture in a CO₂ incubator, the plated cells and Cell Titer-Glo® (Promega #G7571) reagents are brought to room temperature to equilibrate for 30 minutes. 100 μL of Cell Titer-Glo® reagent are added to each well. The plate is shaken for 2 minutes and then left to equilibrate for 10 minutes before determining luminescence on a Tecan GENios microplate reader.

Percent inhibition of cell growth is calculated relative to untreated control wells. All tests are performed in duplicate at each concentration level. The IC₅₀ value for the test agents is estimated using Prism 3.03 by curve-fitting the data using the following four parameter-logistic equation:

$$Y = \frac{Top - Bottom}{1 + {( X / {IC}_{50} )}^{n}} + Bottom$$

where Top is the maximal % of control absorbance, Bottom is the minimal % of control absorbance at the highest agent concentration, Y is the % of control absorbance, X is the agent concentration, IC₅₀ is the concentration of agent that inhibits cell growth by 50% compared to the control cells, and n is the slope of the curve.

Compounds that inhibit the proliferation of one or more leukemia, lymphoma, or solid tumor cell lines in, for example, the MTT cell proliferation assay described by Alley, M. et al. (Cancer Res., 48:589-601, 1988), or in variations of this assay, are herein identified. Appropriate cell lines, MTT cell proliferation assay kits, experimental protocols are available from the American Type Culture Collection (Manassas, Va.).

The peptides below are used to determine their effects on cell proliferation.
SEQ ID NO:13 dR dR dR dR dR dR dR dR dR G dL dE dN dE dT dS dM dL dD dN dL dI dK dR dY dpS G amide; R1 corresponds to a cell penetrating peptide, SEQ ID NO:6, where X is glycine; R2 corresponds to an amino acid sequence derived from c-Myb, SEQ ID NO:8 and an amino acid sequence derived from CREB, SEQ ID NO:11 (using the D-isomer of phosphoserine); R1 and R2 are followed by a terminal spacer, glycine, with a C-terminal amide modification;
SEQ ID NO:14 dR dR dR dR dR dR dR dR dR G dL dE dN dE dT dS dM dL dD dN dL dI dK dR dY pS G amide; R1 corresponds to a cell penetrating peptide, SEQ ID NO:6, where X is glycine; R2 corresponds to an amino acid sequence derived from c-Myb, SEQ ID NO:8 and an amino acid sequence derived from CREB, SEQ ID NO:11 (using the L-isomer of phosphoserine); R1 and R2 are followed by a terminal spacer, glycine, with a C-terminal amide modification;
SEQ ID NO:15 dR dR dR dR dR dR dR dR dR G dL dE dN dE dT dS dM dNIe dD dN dL dI dK dR dY pS G amide; R1 corresponds to a cell penetrating peptide, SEQ ID NO:6, where X is glycine; R2 corresponds to an amino acid sequence derived from c-Myb, SEQ ID NO:8 (where the D-isomer of norleucine has been substituted for a D-isomer of leucine) and an amino acid sequence derived from CREB, SEQ ID NO:11 (using the L-isomer of phosphoserine); R1 and R2 are followed by a terminal spacer, glycine, with a C-terminal amide modification;
SEQ ID NO:16 dR dR dR dQ dR dR dK dK dR G dY G dL dE dN dE dT dS dM dL dL dL dE dL dE dK dI dR amide; R1 corresponds to a cell penetrating peptide, SEQ ID NO:5, where X is glycine; R2 corresponds to an amino acid sequence derived from c-Myb, SEQ ID NO:9; R1 and R2 are immediately followed by a C-terminal amide modification;
SEQ ID NO:17 dR dR dR dR dR dR dR dR dR G dP dA dD dS dS dL dD dN dL dI dK dR dY dpS G amide; R1 corresponds to a cell penetrating peptide, SEQ ID NO:6, where X is glycine; R2 corresponds to an amino acid sequence derived from CREB, SEQ ID NO:12 (using the D-isomer of phosphoserine); R1 and R2 are followed by a terminal spacer, glycine, with a C-terminal amide modification;
SEQ ID NO:18 dR dR dR dR dR dR dR dR dR G dP dA dD dS dS dL dD dN dL dI dK dR dY pS G amide; R1 corresponds to a cell penetrating peptide, SEQ ID NO:6, where X is glycine; R2 corresponds to an amino acid sequence derived from CREB, SEQ ID NO:12 (using the L-isomer of phosphoserine); R1 and R2 are followed by a terminal spacer, glycine, with a C-terminal amide modification.

TABLE 1
Cell Proliferation Assay using Cell Line
THP-1 (SEQ ID NOS: 13, 14 and 15)

| Compound | IC₅₀ values (μM) |
|---|---|
| Cisplatin | 1.6 |
| SEQ ID No: 13 | 17.5 |
| SEQ ID NO: 14 | 13.9 |
| SEQ ID NO: 15 | 14.1 |

As is apparent in Table 1, there are no important differences in the IC₅₀ values for SEQ ID NOS: 13, 14 and 15. Cisplatin data serve as a control.

TABLE 2
Cell Proliferation Assay using Cell
Line THP-1 (SEQ ID NOS: 5 and 16)

| Compound | IC₅₀ values (μM) |
|---|---|
| Cisplatin | 1.6 |
| SEQ ID NO: 5 | >200 |
| SEQ ID NO: 16 | 21.6 |

SEQ ID NO:5 is R1, the cell penetrating peptide component, for SEQ ID NO:16. The IC₅₀ value for SEQ ID NO:5 is much greater than the IC₅₀ value for SEQ ID NO:16. Cisplatin data serve as a control.

### Other Embodiments

Other embodiments will be evident to those of skill in the art. It should be understood that the foregoing detailed description is provided for clarity only and is merely exemplary, not to be limited to any particular methodology, protocol, compound, formulation etc. The spirit and scope of the present disclosure are not limited to the above examples, but are encompassed by the following claims. The contents of all references cited herein are incorporated by reference in their entireties.

What is claimed is:

1. A formulation comprising a peptide comprising R1 and R2, wherein R1 is a cell penetrating peptide, and R2 is a peptide derived from a group consisting of: the sequence of c-Myb, the sequence of CREB, and a sequence comprising both a peptide sequence derived from the sequence of c-Myb and a sequence derived from the sequence of CREB.

2. The formulation of claim 1, wherein R1 comprises a sequence that is about 80% identical to SEQ ID NO:5 or SEQ ID NO:6.

3. The formulation of claim 2, wherein R1 comprises a sequence that is identical to SEQ ID NO:5 or SEQ ID NO:6.

4. The formulation of claim 1, wherein R2 comprises a sequence that is about 80% identical to a sequence selected from the group consisting of SEQ ID NOS:7-9.

5. The formulation of claim 4, wherein R2 comprises a sequence that is identical to a sequence selected from the group consisting of SEQ ID NOS:7-9.

6. The formulation of claim 1, wherein R2 comprises a sequence that is about 80% identical to a sequence selected from the group consisting of SEQ ID NOS:10-12.

7. The formulation of claim 6, wherein R2 comprises a sequence that is identical to a sequence selected from the group consisting of SEQ ID NOS:10-12.

8. The formulation of claim 1, wherein the peptide comprises a sequence that is about 80% identical to a sequence selected from the group consisting of: SEQ ID NOS:13-18.

9. The formulation of claim 8, wherein the peptide comprises a sequence that is identical to a sequence selected from the group consisting of: SEQ ID NOS:13-18.

10. The formulation of claim 1, wherein the peptide is comprised entirely of D-amino acids.

11. The formulation of claim 1, wherein the peptide is comprised of a mix of D-amino acids and L-amino acids.

12. The formulation of claim 1, wherein the peptide comprises one or more non-naturally occurring amino acids.

13. The formulation of claim 1, wherein the peptide comprises one or more synthetic amino acids.

14. The formulation of claim 1, further comprising one or more pharmaceutically acceptable excipients.

15. The formulation of claim 1, wherein R1 occurs at the N- or C-terminus of the peptide.

16. The formulation of claim 1, optionally comprising one or more amino acids at the termini of the peptide and/or between R1 and/or R2.

17. The formulation of claim 16, wherein the peptide comprises an amino acid with a C-terminal amide modification.

18. A method of treating or preventing a disease or disorder associated with β-hemoglobin, CBP or p300 misregulation, comprising administering a therapeutically effective amount or a formulation comprising a peptide comprising R1 and R2, wherein R1 is a cell penetrating peptide, and R2 is a peptide derived from a group consisting of: the sequence of c-Myb, the sequence of CREB, and a sequence comprising both a peptide sequence derived from the sequence of c-Myb and a sequence derived from the sequence of CREB, or a pharmaceutically acceptable salt thereof.

19. The method of claim 18, wherein the disease or disorder is selected from the group consisting of: acute leukemia, chronic leukemia, myeloproliferative disorders, lymphoma, solid tumors, sickle cell disease and β-thalassemia.

20. The method of claim 18, wherein the therapeutically effective amount of the peptide is a dose related to total body mass, wherein the dose is selected from the group consisting of: about 0.01 mg/kg/day to about 100 mg/kg/day; about 0.01 to about 0.03 mg/kg/day; about 0.03 to about 0.1 mg/kg/day; about 0.1 to about 0.3 mg/kg/day; about 0.3 to about 1 mg/kg/day; about 1 to about 3 mg/kg/day; about 3 to about 10 mg/kg/day; about 10 to about 30 mg/kg/day; and about 30 to about 100 mg/kg/day.

21. The method of claim 18, wherein the therapeutically effective amount of the peptide is a dose related to total body surface area, wherein the dose is selected from the group consisting of: about 0.3 to 3000 mg/m²/day; about 0.3 to about 1 mg/m²/day; about 1 to about 3 mg/m²/day; about 3 to about 10 mg/m²/day; about 10 to about 30 mg/m²/day; about 30 to about 100 mg/m²/day; about 100 to about 300 mg/m²/day; about 300 to about 1000 mg/m²/day; and about 1000 to about 3000 mg/m²/day.

22. The method of claim 18, wherein R1 comprises a sequence that is about 80% identical to SEQ ID NO:5 or SEQ ID NO:6.

23. The method of claim 22, wherein R1 comprises a sequence that is identical to SEQ ID NO:5 or SEQ ID NO:6.

24. The method of claim 18, wherein R2 comprises a sequence that is about 80% identical to a sequence selected from the group consisting of: SEQ ID NOS:7-9.

25. The method of claim 24, wherein R2 comprises a sequence that is identical to a sequence selected from the group consisting of: SEQ ID NOS:7-9.

26. The method of claim 18, wherein R2 comprises a sequence that is about 80% identical to a sequence selected from the group consisting of: SEQ ID NOS:10-12.

27. The method of claim 26, wherein R2 comprises a sequence that is identical to a sequence selected from the group consisting of: SEQ ID NOS:10-12.

28. The method of claim 18, wherein the peptide comprises a sequence that is about 80% identical to a sequence selected from the group consisting of: SEQ ID NOS:13-18.

29. The method of claim 28, wherein the peptide comprises a sequence that is identical to a sequence selected from the group consisting of: SEQ ID NOS:13-18.

30. The method of claim 18, wherein the peptide is comprised entirely of D-amino acids.

31. The method of claim 18, wherein the peptide is comprised of a mix of D-amino acids and L-amino acids.

32. The method of claim 18, wherein the peptide comprises one or more non-naturally occurring amino acids.

33. The method of claim 18, wherein the peptide comprises one or more synthetic amino acids.

34. The method of claim 18, wherein the formulation further comprises one or more pharmaceutically acceptable excipients.

35. The method of claim 18, wherein R1 occurs at the N- or C-terminus of the peptide.

36. The method of claim 18, optionally comprising one or more amino acids at the termini of the peptide and/or between R1 and/or R2.

37. The method of claim 18, wherein the peptide comprises an amino acid with a C-terminal amide modification.
